(19) United States
(12) Patent Application Publication
(10) Pub. No.: US 2010/0121356 A1
(43) Pub. Date: May 13, 2010

(54) Apparatus for Minimum Invasive Fasciectomy

(75) Inventors: Rolf Hartmann, Boras (DE); Martin Russlies, Luebeck (DE); Rudolf Ascherl, Leipzig (DE); Wolfgang Koller, Luebeck (DE)

Correspondence Address: DIEDERIKS & WHITELAW, PLC, 13885 HEDGEWOOD DR., SUITE 317, WOODBRIDGE, VA, 22193, US

(73) Assignee: UNIVERSITAETSKLINIKUM SCHLESWIG-HOLSTEIN, Luebeck (DE)

(21) Appl. No.: 12/373,930

(22) PCT Filed: Jul. 10, 2007
(86) PCT No.: PCT/DE07/01221

§ 371 Date: Dec. 15, 2009

(30) Foreign Application Priority Data
Jul. 15, 2006 (DE) 10 2006 032 897.3

(51) Int. Cl. A61B 17/08

(52) U.S. Cl. 606/151

(57) ABSTRACT

Apparatus for minimum invasive fasciectomy, characterized by a tubular fixation element, including two detachably interconnected semitubular shells with in each case a wedge-shaped recess formed at a proximal end and at a distal end, a deflecting bar, whose length essentially corresponds to the portion between the recesses, and lifting wedges with in each case receptacles located at the lower portion thereof for the detachable connection of the lifting wedges to the deflecting bar, wherein the titling wedges can be introduced into the wedge-shaped recesses, accompanied by a sliding apart of the detachably interconnected semitubular shells.

### CROSS-REFERENCE TO RELATED APPLICATIONS

This application represents a National Stage application of PCT/DE2007/001221 entitled “Minimally Invasive Fascietomy Device” filed Jul. 10, 2007, pending.

### BACKGROUND OF THE INVENTION

The invention relates to an apparatus for minimum invasive fasciectomy with subsequent closing of the opening in the tissue layer using a surgical suture thread.

The fascia is a slightly expandable envelope of individual organs, muscles or muscle groups. Body fascias envelop the total musculature of the torso or extremities. In the case of a weakening of the fascial tissue it is no longer able to fulfil its supporting function. Consequently there can be a protrusion of the supporting tissue, referred to as herniation. In certain circumstances surgical intervention is necessary for removing this functional restriction.

Methods are known which retighten or reinforce the fascia by gathering up or doubling, so that it restores its retaining function. Fascial material is also used in many operative procedures as transplant (transplanted tissue pieces) in different shapes and sizes. The tissue is preferably suitable for replacement, reconstruction, suspension, interposing and occluding tissue defects.

The indicated operative methods for the reconstruction or removal of fascia are usually so-called open methods, i.e. the skin and hypodermis over the entire operating area are opened (Seybold, K.: Die Augmentationsnaht des vorderen Kreuzbandes mit einer Kordel oder einem Fascia lata-Streifen, Munich University, 1994 dissertation; Gohrbrandt E. et al., Handbuch der Chinirgie, Berlin/de Gruyter, 1965). This involves a corresponding traumatization of the surrounding tissue. To reduce this tissue damage, such as arises in conventional operative procedures, minimum invasive methods have been evolved in surgery. One example of fasciectomy is so-called fascia strippers. The starting part of the transplant to be removed is freely prepared by means of a small skin cut, in which the fascia stripper is inserted and is then advanced under the skin into the desired position of the taenia. This operating method can be performed in very varied form, but essentially corresponds to those of the known disclosures (DE/EP 0 707 456 T 1, DE 695 33 893 T 2). A cutting mechanism to be operated on the instrument handle then separates the fascia on the stripper start under the skin.

However, a major disadvantage of this minimum invasive method is that the resulting fascia gap cannot be reclosed and consequently a hernia can result. In addition to cosmetic damage, discomfort due to the strangulated musculature can occur.

For all the prior art operating methods the same problem arises, namely in a minimum invasive removal method with subsequent fascia closer the most serious difficulty is that a muscular herniation occurs prior to the making of the fasciopphaphy or the cutting edges of the fascia cannot be readapted due to the high tissue tension. The most varied devices with the most varied handling procedures are also known for closing the opening. The most widespread is known from DE 199 44 236 A1. Moreover, the disclosures of DE 199 44 236 A1, DE 200 09 815 U1 and DE 695 24 130 T2 cover a very broad functional range of different closure methods.

### SUMMARY OF THE INVENTION

The problem of the invention is to provide an apparatus preventing the moving apart of the fascia cutting edges before fasciopphaphy is carried out, so as to allow a minimum invasive fascia adaptation or removal with a following fasciopphaphy.

The problem is solved by means of the apparatus for minimum invasive fasciectomy, characterized by a tubular fixation element comprising two detachably interconnected semitubular shells with in each case a wedge-shaped recess formed at a proximal end and at a distal end, a deflecting bar, whose length essentially corresponds to the portion between the recesses and lifting wedges with receptacles in each case located an the lower portion thereof for detachable connection of the lifting wedges to the deflecting bar, wherein the lifting wedges can be introduced into the wedge-shaped recesses accompanied by the sliding apart of the detachably interconnected semitubular shells.

### BRIEF DESCRIPTION OF DRAWINGS

The invention is described in greater detail hereinafter relative to the attached drawings, wherein show:

FIG. 1 A cross-sectional view of the fixation principle.

FIGS. 1a-1d Cross-sectional views of the fixation arrangement in various stages of a removal process.

FIG. 2 A semitubular fixation element.

FIGS. 2a-2d Cross-sectional views of closure apparatuses.

FIG. 3 An exploded view of deflecting bar, semitubular fixation units, closure apparatus, and lifting wedges.

FIGS. 3a-3d Side views of lifting wedges in operation.

FIGS. 4a-4d The working steps in the working channel.

FIG. 5 The spiral capillary tube with grip or handle.

### DETAILED DESCRIPTION OF INVENTION

Prior to the use of the apparatus according to the invention, the surface of the fascia portion intended for gathering up or removal is projected onto the skin. Over the short sides of the fascia rectangle in the smallest possible manner the skin and hypodermis 5 are divided and the fascia 4 exposed, followed by the cutting of said fascia 4 corresponding to the short sides of the fascia rectangle. Using a spatula-like instrument the fascia area between the skin cuts are now separated from the hypodermis 5. A deflecting bar 1 is introduced through the fascia cut under the fascia 4 and advanced in the direction of the second fascia cut 3. Two semitubular fixation units 2 are introduced through the skin cut over fascia 4 or under the skin and advanced in the direction of the second skin cut 3. The fixation principle is diagrammatically represented in the cross-sectional view with the deflecting bar 1 introduced beneath the fascia 4 and the two semitubular elements 2 located under the skin and above the fascia 4.

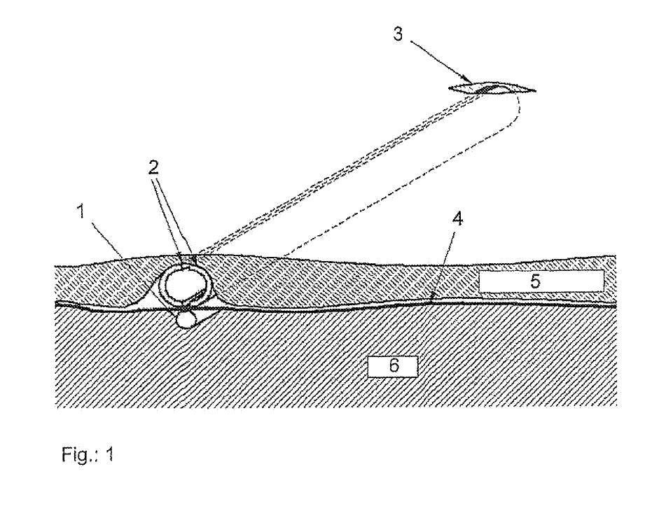

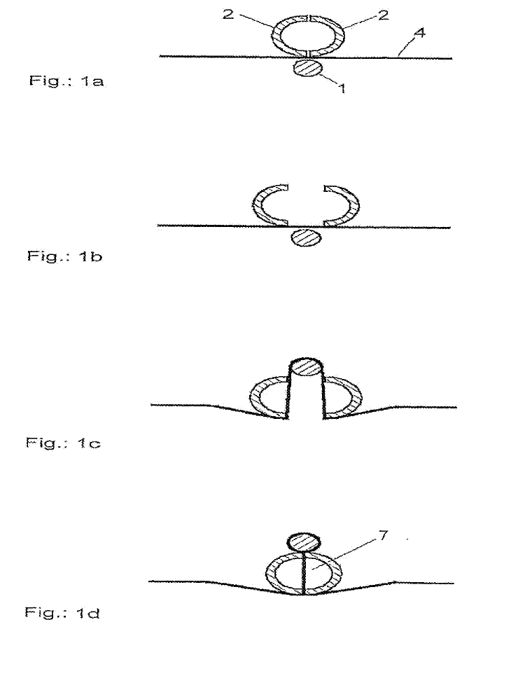

FIGS. 1a to 1d show the individual partial steps of the removal process. Following the separation of the fascia 4 the fixation elements 2 are subcutaneously introduced along the deflecting bar 1, previously placed under the fascia 4, at the distal skin cut. In this state the semitubular fixation elements 2 are closed and form a tubular device. The deflecting bar 1 is introduced under the fascia 4. The main closure apparatus 8 is held together by two hollow threaded pins. By means of a hexagon socket wrench the main closure apparatus 8 can be opened, so that the fixation elements 2 move away from one another in parallel. Following the opening of the device the deflecting bar 1 is positioned between the two semitubular fixation elements 2 and mounted on the lifting wedges 11, 12 introduced at the distal skin cut and the second skin cut 3. After the two fixation elements 2 have moved apart, cf. FIG. 1b, the deflecting bar 1 moves upwards relative to the fascia plane 4 and the fixation elements 2 move downwards. When the tissue piece to be gathered up or cut out by the deflecting bar 1 has been extracted from the fascia plane 4, the fixation elements 2 move towards one another again and consequently bring the future cutting edges into contact, as is diagrammatically shown in FIG. 1c. FIG. 1d diagrammatically shows the creation of a working channel 7 for fasciectomy, in that the semitubular fixation elements 2 used for fixation are so designed that they give an approximately circular lumen when brought together with interposed fascia 4. The resulting tube internal diameter now serves as the working channel 7. As the other soft parts are located outside the tube internal diameter, the further working steps can take place without visual monitoring.

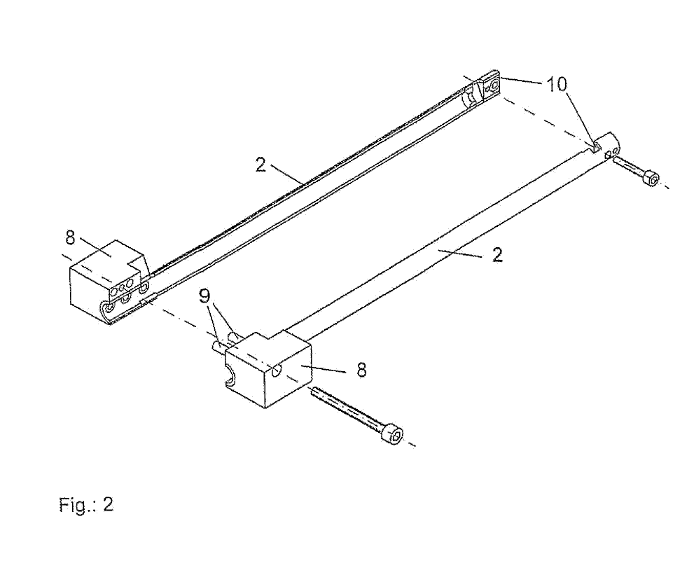

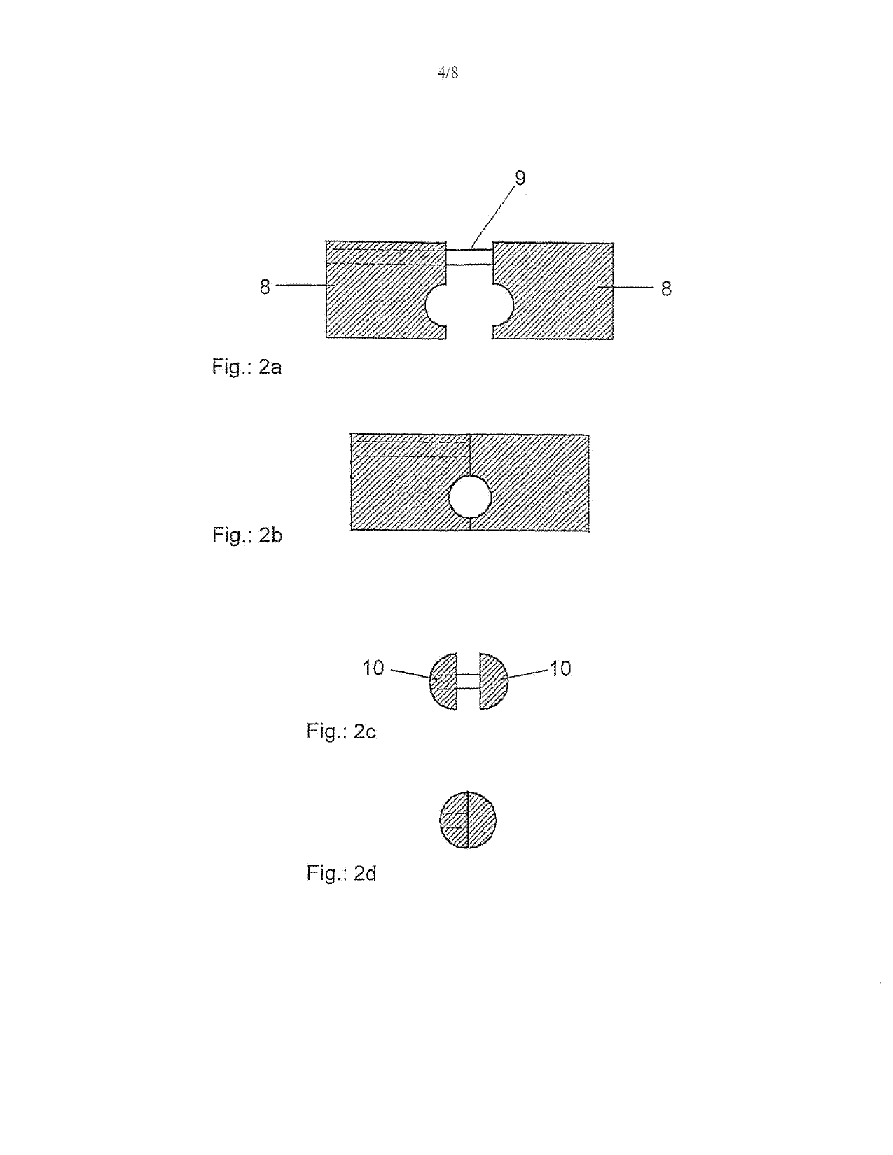

To ensure the function of the fixation System, the semitubular fixation elements 2 must be moved parallel to one another. This is made possible by a closure apparatus 10 at both ends of the device, which links the semitubular fixation elements 2 to a device unit as the main closure apparatus 8. As the cutting edges of the fixation elements 2 must precisely strike one another, the main closure apparatus 8 is equipped with sliding pins 9 as guide elements. The closure principle of the closure apparatuses at the device ends is illustrated in the cross-sectional views of FIGS. 2a to 2d. As shown in FIG. 2 and FIGS. 2a to 2d, the closure apparatuses are differently dimensioned. The device end, which is introduced first via the first skin cut beneath the fascia, includes a small closure unit 10 accessible via the second skin cut. At the other device end is located the main closure apparatus 8, which is guided by two sliding pins 9 and moved with the aid of a threaded rod. Due to its size this part of the device remains positioned outside the skin. It simultaneously serves as a device handle. The cross-sections in FIGS. 1a to 1c show the units in the opened and closed state.

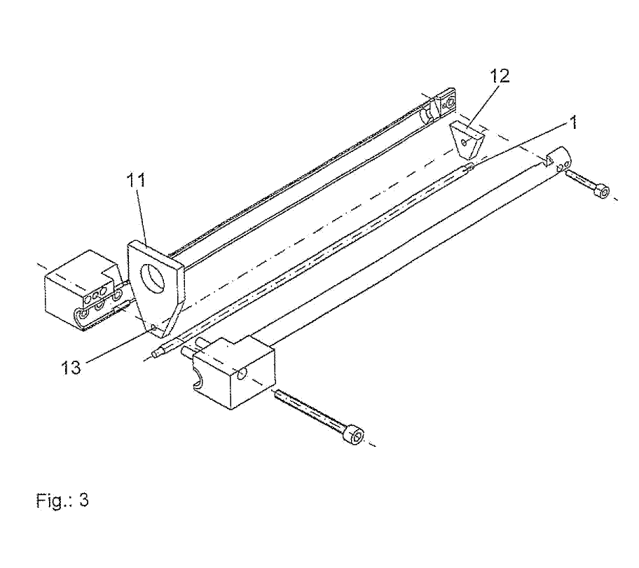

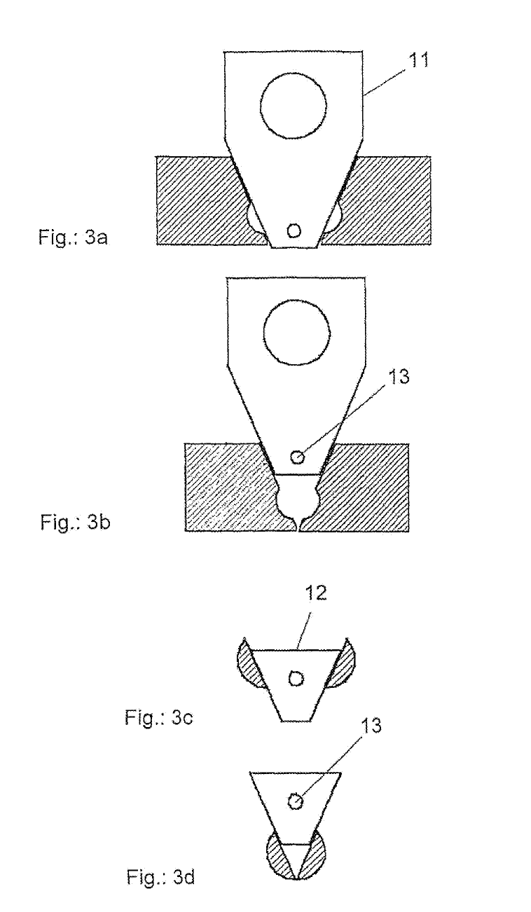

The lifting principle of the deflecting bar 1 functions by means of inclined planes, which transform the closure movement, i.e. the sliding onto one another of the semitubular fixation elements 2, into a lifting movement. On the milled ends of the deflecting bar 1 are engaged wedge-like attachments as lifting wedges 11, 12 and are placed in the lifting shafts at both ends of the device, as shown in FIG. 3. On device closure the wedge-like attachments 11, 12 slide upwards into the shafts and with the same the deflecting bar 1 with the fascia 4 is raised upwards. The lifting principle of the deflecting bar 1 is visible in a cross-sectional view of the wedge attachments 11, 12 in the lifting shafts according to FIGS. 3a to 3d. The wedge attachments 11, 12 are forced upwards on closing the closure units. The deflecting bar held in the small holes 13 is raised upwards between the semitubular fixation units 2. The cross-sectional views of FIGS. 3a 3b and 3c, 3d show the units in the opened and closed state.

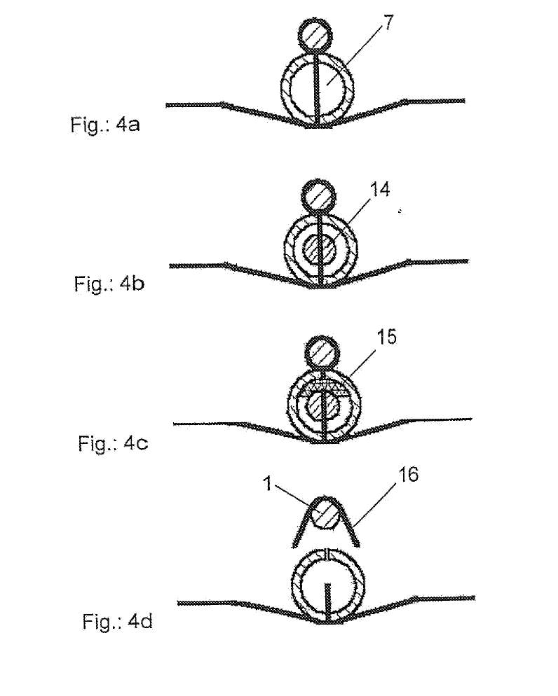

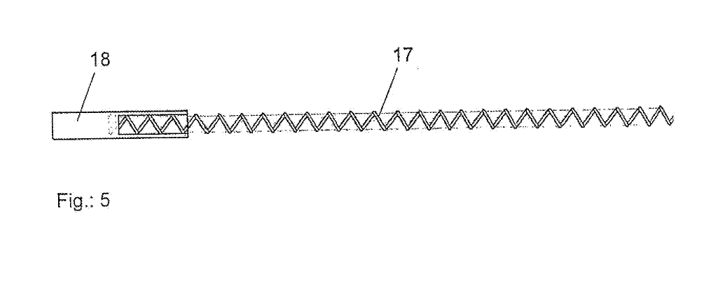

The further working sequence for the treatment of the fascia 4 is diagrammatically illustrated in FIGS. 4a to 4d and takes place in working channel 7, corresponding to FIG. 4a. Far further splinting a fork-like structure 14 with a fork leg to the left and right of the fascia 4 is introduced centrally into the working channel 7, cf. FIG. 4b. As a further working step a scalpel slide 15 is introduced above said splint into the working channel 7, as shown in FIG. 4c. On advancing the scalpel onto the fork, the fascia is cut through in the upper area of working channel 7 and the piece which runs round the deflecting bar 1 in the form of taenia 16 becomes free, cf. FIG. 4d. The taenia 16 can now be removed together with the deflecting bar 1. The suture of the still adapted fascia 4 takes place by means of a spiral capillary tube 17 according to FIG. 5. The capillary tube 17 on the splinting 14 is fully advanced up to the other device end. In the internal diameter of the capillary tube is introduced a fascia fibre and is gripped at the other end. By turning back the spiral by means of a handle 18 the fascia fibre remains as a continuous, coiled over suture in the fascia 4. Subsequently the suture projections are sutured to the intact fascia 4, after which further wound closure takes place.

Thus, the inventive apparatus is suitable both for a gathering up operation and also for a transplant removal.

1. Apparatus for minimum invasive fasciectomy comprising:
a tubular fixation element comprising two detachably interconnected semitubular shells with in each case a wedge-shaped recess formed at a proximal end and at a distal end,
a deflecting bar, whose length essentially corresponds to the portion between the recesses and
lifting wedges with receptacles in each case located an the lower portion thereof for detachable connection of the lifting wedges to the deflecting bar,
wherein the lifting wedges can be introduced into the wedge-shaped recesses accompanied by the sliding apart of the detachably interconnected semitubular shells.

2. Apparatus according to claim 1, characterized in that the lifting wedges have different dimensions.

3. Apparatus according to claim 1, characterized in that the semitubular shells are detachably interconnected by means of a threaded pin.

4. Apparatus according to claim 1, characterized in that the interconnected semitubular shells form a working channel.

5. Apparatus according to claim 4, characterized in that the working channel is set up for receiving a fork rail.

6. Apparatus according to claim 4, characterized in that the working channel is set up for receiving a spiral capillary tube which receives a fascia fibre.

7. Apparatus according to claim 5, characterized in that the working channel is set up for receiving a spiral capillary tube which receives a fascia fibre.
